(19) United States
(12) Patent Application Publication
(10) Pub. No.: US 2012/0070506 A1
(43) Pub. Date: Mar. 22, 2012

(54) Animal Treatment

(75) Inventor: Wayne Frederick Leech, Auckland (NZ)

(73) Assignee: BOMAC RESEARCH LIMITED, Manukau City, Auckland (NZ)

(21) Appl. No.: 13/145,341

(22) PCT Filed: Dec. 18, 2009
(86) PCT No.: PCT/NZ2009/000299

§ 371 Date: Aug. 16, 2011

(30) Foreign Application Priority Data
Jan. 20, 2009 (NZ) 574317

(51) Int. Cl. A61K 35/56; A61P 29/00; A61P 19/02

(52) U.S. Cl. 424/547

(57) ABSTRACT

The present invention relates to a composition for an animal within a particular weight range including at least one non-steroidal anti-inflammatory drug (NSAID) compound and at least one mussel extract characterised in that the therapeutic dose of NSAID compound in the composition is substantially less than the usual therapeutic dose.

### TECHNICAL FIELD

The present invention relates to improved animal treatment. In particular, the present invention provides an alternative therapy for the treatment or prevention of connective tissue diseases and the symptoms thereof for animals such as dogs, cats and horses.

### BACKGROUND ART

The connective tissues surrounding skeletal joints of human and non-human animals are constantly subject to stresses and strains from mechanical forces that can result in afflictions such as arthritis (rheumatoid and osteoarthritis), joint inflammation and joint stiffness. This is the same for both human and non-human animals and tends to be more prevalent in older age. The causes behind rheumatoid and osteoarthritis are different. Rheumatoid arthritis is characterised as a systemic autoimmune disease affecting both the joints and other soft tissues. Osteoarthritis results from deterioration of cartilage which may result in local inflammation of the joint(s). The majority of non-human animals, and particularly companion animals, tend to be afflicted predominantly by osteoarthritis.

In osteoarthritis, cumulative stress in the joints results in loss of integrity of the cartilage matrix. Resulting damage causes acceleration in deterioration of cartilage and synovial fluid. The body has a natural capability to restore and synthesise normal collagen structures but with aging, there is a decreased ability to restore collagen. In the case of osteoarthritis, symptoms of the condition include pain in the joint, redness, heat, joint effusion, joint oedema and loss of joint function.

The treatment of connective tissue diseases in both humans and non-humans can be problematic.

It is usually not an option for the sufferer to stop normal movements associated with day to day activities. Consequently, treatment is often directed to the symptoms of the disease and not the underlying cause.

Typical drug treatments include use of steroids such as corticosteroids and other anti-inflammatory materials such as high doses of aspirin for humans. In veterinary drug treatments, hyaluronic acid and polysulfonated glycosaminoglycan are used, particularly for equines to reduce connective tissue pain and swelling. Unfortunately, almost all of these remedies lose their effectiveness over time and also have side effects such as stomach ulceration, platelet deactivation and/or decreased blood supply to the kidney.

More recently, non-steroidal anti-inflammatory drugs (NSAIDs) have been developed that specifically inhibit cyclooxygenase-2 (COX-2) activity without inhibiting cyclooxygenase-1 (COX-1) activity. Such NSAID's result in fewer gastro-intestinal complication side effects common with non-selective NSAID's.

NSAID activity including the characteristics of COX-1 and COX-2 enzymes are extensively described in the prior art, for example the article ‘Practical COX-1 and COX-2 Pharmacology: What's it all About?’ by C. Jones.

By way of example, one COX-2 selective drug remedy widely used for treatment of joint problems is carprofen, sold by Pfizer under the trade name Rimadyl® with related patents U.S. Pat. No. 4,264,500 and U.S. Pat. No. 6,013,808.

Carprofen has the formula:

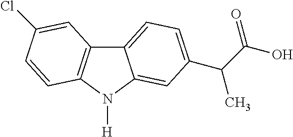

Carprofen is a non-steroidal anti-inflammatory drug (NSAID) of the propionic acid class that includes ibuprofen, maproxen and ketoprofen. Carprofen is the non-proprietary designation for a substituted carbozole, 6-chloro-alpha-methyl-9H-carbozole-2-acetic acid.

Carprofen has been widely used in veterinary treatments as it is a selective COX-2 inhibitor and has only limited side effects in treatment of animals. These side effects can however be serious, with hepatotoxicity (acute hepatic necrosis) or other liver damage a known adverse effect of carprofen.

Unfortunately, prolonged usage of carprofen is often out of reach due to its cost (approximately $US1.00 per tablet usually taken twice daily) and fears, whether reasoned or unreasoned of side effects of treatment. Ultimately, carprofen merely treats the symptoms rather than treating the underlying cause: in the case of osteoarthritis carprofen does not rebuild degenerated cartilage.

Alternative natural product therapies have also been considered for treatment of osteoarthritis, joint problems and joint inflammation.

Natural products have frequently been the source of effective drugs and recently there has been an increased interest in the analysis of these natural products, especially if a clinical benefit can be established.

Natural product compounds that can assist in joint remedies include orally administered chondroprotective agents, glucosamine and chondroitin sulphate, which in the body are or are used to produce important constituents of cartilage. There are reports that these substances have beneficial effects in humans however, there are few reports in relation to veterinary applications.

Certain marine organisms contain compounds that, when administered to humans and non-human animals have been shown to aid in the alleviation of inflammation. One of these organisms is the mussel and more specifically, Perna canaliculus (Green lipped or green shell mussel).

Rainsford and Whitehouse, 1980, showed that freeze-dried powder preparation of the whole mussel given orally to rats showed modest anti-inflammatory effect. Another study (Korthauer and Delatorre 1992) found that the oral administration of a glycosaminoglycan extracted from Perna canaliculus to dogs with arthritis alleviated signs of lameness or faulty posture.

WO 96/05164 discloses that lipid extracts of Perna canaliculus and Perna canaliculus have an active component exhibiting anti-inflammatory activity.

WO 00/56164 describes a pet food that incorporates a supplemental amount of mussel extract for the purposes of alleviating joint inflammation.

Combination therapies have also been considered. For example, the applicant's related application, WO 01/05411 describes a particular combination of natural anti-inflammatory components including Perna canaliculus, shark cartilage and use of enhancing agents including a bark extract rich in antioxidant compounds.

A problem noted from use of mussel-derived supplements is that the therapy takes time to show results. Korthauer and Delatorre 1992 noted that it took 8 weeks for the mussel extract therapy to show the desired results. Similarly, in the applicants related application WO 01/05411, it took 1 to 2 months before the animals' condition stabilised. In the interim time period between stabilisation and initial therapy the animal still exhibits, to a lessening extent over time, symptoms of pain, swelling and limitation of movement.

Thus it is an object of the present invention to provide an alternative therapy that addresses the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### DISCLOSURE OF INVENTION

According to one aspect of the present invention there is provided a composition for an animal within a particular weight range including at least one NSAID compound and at least one mussel extract

characterised in that
the therapeutic dose of NSAID compound in the composition is substantially less than the usual therapeutic dose.

According to a further aspect of the present invention there is provided a method of treatment or prevention of a connective tissue disease or the symptoms thereof in an animal, characterised by the step of administering to the animal a composition containing a therapeutically effective amount of at least one NSAID compound and mussel extract,

wherein the therapeutic dose of NSAID compound is substantially less than the usual therapeutic dose.

According to a further aspect of the present invention there is provided the use of a therapeutically effective amount of at least one NSAID compound and mussel extract, wherein the therapeutic dose of NSAID compound is substantially less than the usual therapeutic dose in the manufacture of a medicament for the treatment or prevention of a connective tissue disease or the symptoms thereof.

According to a further aspect of the present invention there is provided the use of at least one NSAID compound and at least one mussel extract, wherein the therapeutic dose of NSAID compound is substantially less than the usual therapeutic dose for the treatment or prevention of a connective tissue disease or the symptoms thereof in an animal.

The term ‘NSAID’ should be taken as meaning any non-steroidal anti-inflammatory drug.

Preferably, the NSAID compound is a cyclooxygenase (COX-2) selective NSAID compound. More preferably, the COX-2 selective NSAID compound is selected from: carprofen, maproxen, ibuprofen, ketoprofen, piroxicam, diclofenac, etodolac, flunixin, deracoxib, meloxicam, celecoxib, rofecoxib, and combinations thereof. Most preferably, the COX-2 selective NSAID compound may be carprofen.

For the purposes of this specification the term ‘mussel’ refers to any of several marine bivalve molluscs, especially edible members of the family Perna and Mytilus.

Preferably, the mussel may be of Perna or Mytilus genus. Most preferably, the mussel may be Pema canaliculus.

The term ‘extract’ should be taken as meaning a preparation of one or more components derived (i.e. separated away) or processed from at least one mussel.

More preferably, the term ‘extract’ refers to either a powdered form of the mussel meat (not including the shell) or a concentrated preparation of the lipid portion of the mussel meat. However, it should be appreciated that the term ‘extract’ also encompasses other powdered or liquid forms of the mussel meat or a specific portion or portions thereof.

The term ‘therapeutic dose’ should be taken as meaning an amount of active ingredient administered to a subject in a given time frame that is intended to result in a medical outcome indicated by animal trials and the like.

The usual therapeutic dose of NSAID is at least 2 mg NSAID/kg/day. A dose less than this is usually considered not to be sufficient to provide effective treatment for inflammatory diseases.

The inventors have unexpectedly found that the beneficial synergistic effect of the NSAID compound in combination with mussel extract allows the dose of NSAID compound to be decreased to levels lower than currently available in NSAID formulations whilst still providing a similar therapeutic effect to formulations which deliver ≧2 mg NSAID/kg/day.

The reduced therapeutic dose of NSAID has a number of important advantages. For example, it may help avoid the side effects associated with NSAIDs (for example, hepatoxicity (acute hepactic necrosis), gastric damage or liver damage). Also the active ingredient NSAID is quite expensive to provide. A lower therapeutic dose of NSAID therefore ultimately means cheaper production costs which will lower the cost of treatment for the consumer.

Previously, the applicant disclosed a formulation containing both NSAID and mussel extract (NZ 534552). Here, they showed a synergistic effect within a formulation containing both NSAID and mussel extract. The strong anti-inflammatory effects of a NSAID compound complement the anti-inflammatory and cartilage recuperative effects associated with mussel extract. The fast acting anti-inflammatory effects of a NSAID compound are believed to allow the mussel extract's cartilage regenerative effects to take place sooner than if the mussel extract were administered alone. The applicants also un-expectantly found the synergistic effect is still present when the NSAID compound is between 0.5 and 2.0 mg/kg/day, whereby the fast acting NSAID still allows the mussel extract effects to occur sooner than if the mussel extract is administered alone.

In NZ 534552, the applicant also found that the side effects associated with NSAIDs are reduced in the combination formulation. This is partially because the NSAID duration period may be shortened due to the quicker response time of the mussel extract. Furthermore, the mussel appeared to provide protection to the gastrointestinal tract, thus preventing ulceration and alleviating detrimental effects on the liver. These positive attributes are still present in the current formulation.

However, the applicant did not previously discover that the synergistic effect of NSAID with mussel extract could permit the dose of NSAIDs to be lowered beyond levels previously tested yet maintain the therapeutic effect. All proven formulations to date utilize a recommended dose of at least 2 mg/kg/day. Often the dosage is double this (4 mg/kg/day) or more.

Although NSAID treatment does work effectively in masking the symptoms associated with inflammatory diseases, it does have significant and detrimental side effects including hepatotoxicity (acute hepatic necrosis) or other gastric damage or liver damage. Therefore any formulation which has a reduced dosage of COX-2 selective NSAIDs, yet still has the desired effect in comparison to current formulations is advantageous over formulations currently used as it will help avoid hepatoxicity (acute hepatic necrosis), gastric damage or liver damage.

Trials performed by the applicant show that the rate and severity of side-effects tended to increase when the formulation's dosage of NSAIDs is above 2 mg/kg/day.

Further, the applicant has found that, to permit decreased levels of NSAID in the formulation while still providing a similar therapeutic effect to higher doses (e.g. 2-4 mg NSAID per mg/kg/day), the minimum amount of mussel extract is approximately 5 fold the amount (w/v) of NSAID present in the formulation.

If mussel extract is below 5 fold the amount of NSAID, the level of NSAID must be raised above 2 mg/kg/day to provide a therapeutic response.

Preferably, the maximum amount of mussel extract is approximately 20 mg/kg/day. The applicants acknowledge that there is unlikely to be a maximum level where the NSAID begins to show a decreased therapeutic effectiveness. However, out of convenience, the applicant foresees that a level above 20 mg/kg/day is unlikely to provide substantial advantages that outweigh the cost in production, etc. However, levels of mussel extract beyond 20 mg/kg/day should not be considered beyond the scope of the invention.

Preferably the connective tissue disease treated or prevented is selected from: osteoarthritis, joint inflammation, cartilage degradation, hip dysplasia, or combinations thereof.

The term ‘symptom’ refers to any subjective evidence of disease or patient's condition.

Preferably, the composition may be used for treatment of both the underlying cause and symptoms associated with connective tissue disease. Most preferably, the symptoms may include pain and reduced mobility of the joint and the underlying cause is primarily due to degradation of cartilage from the joint. However, this should not be seen as limiting as it should be appreciated by those skilled in the art that, due to the potent anti-inflammatory nature of the composition, other inflammation related disorders may also be able to be treated using the composition of the present invention such as rheumatoid arthritis inflammation. These include strained ligaments or muscles, or for postoperative use to control pain associated with soft tissue and orthopaedic surgeries. In one preferred embodiment, the composition of the present invention is used for treatment of hip dysplasia.

In preferred embodiments, the composition may be in a form suitable for oral administration. For example, oral administration can be achieved using tablets, capsules, drinks, tonics, sublingual wafer or food ingredients. Most preferably, the composition may be formulated for oral administration in the form of a tablet or capsule. This should not be seen as limiting as it should be appreciated by those skilled in the art that other (non-oral) formulations may be possible including injectable formulations, suspensions, powders, implants, transdermal patches, boluses, suppositories, topical creams/gels and the like. It should be acknowledged that the applicant envisions that any administration method could be used as long as the method supplies the required therapeutic dose.

Most preferably, the composition may further include: carriers, diluents, fillers, flavourings, colourings, excipients, modifiers, humectants, stabilisers, emulsifiers, and other known formulation components.

In preferred embodiments, the animal to be treated may be a companion animal. More preferably, the animal may be from the species canine, feline or equine. Most preferably, the animal may be a domestic dog. However, this should not be seen as limiting as it should be appreciated by those skilled in the art that based on previous results found for NSAID compounds when used alone and mussel extracts when used alone, the combination therapy of the present invention may be used in other animals, including humans. For example, carprofen has been used to treat cats, rabbits, chinchillas, rats and birds.

It should further be appreciated by those skilled in the art that dose rates of both NSAID compound and mussel extract may vary accordingly depending on the metabolism level, age, gender, species or genetics of the animal and other biochemical factors, such as seasonal dietary requirements.

It is envisaged that the composition of the present invention may be used as an effective ‘first line of treatment’ for connective tissue afflictions including joint inflammation, osteoarthritis and/or cartilage degradation. This is due to the synergistic effects of the combination stabilising the affliction quickly and addressing both the symptoms of the affliction (inflammation) and the underlying cause (cartilage degradation).

Following treatment by the composition of the present invention, over an initial period (e.g. two weeks), it is envisaged by the applicant that a sufferer could shift to a stabilising treatment such as that described in the applicant's publication WO 01/05411 after treatment with the composition of the present invention.

Alternatively, because of the reduced dose, a patient may suffer little or no side effects such that they wish to continue the same treatment instead of changing to a treatment such as that described in the applicant's publication WO 01/05411.

It should be appreciated by those skilled in the art that advantages gained from preferred embodiments of the composition of the present invention may include but are not limited to:
A reduced dose of NSAID. This was shown by the inventors to maintain a therapeutic effect, yet reduce the side effects observed when the dose is increased to levels currently used in the field (>2 mg per kg per day).
Due to the potentially reduced side-effects, an animal may continue this treatment plan gaining from the beneficial effects of the synergistic interaction and activity of the two active components in the formulation.
The protective effects of mussel extracts identified in NZ 534552 are maintained in the current formulation.
The synergistic effects between NSAID and the mussel extract identified in NZ 534552 are maintained in the current formulation. In other words, the delay time associated with prior art mussel extracts used for treating joint inflammation is effectively removed or at least significantly reduced.
NSAID compounds may only be used for a short duration if so desired. Some NSAID studies have shown that NSAID drugs may exhibit reduced effectiveness over time hence the short duration and low dose of use in the present invention could be desirable to avoid this potential reduced effectiveness overtime.
The cost of treatment may be reduced as the NSAID compound (which contributes to this cost) is lowered in the current formulation.
Using the mussel extract as the major component (w/w), the present invention takes advantage of a more ‘natural’ alternative, which are increasingly becoming preferred by human patients or human pet-animal owners in many medical treatments.

### BEST MODES FOR CARRYING OUT THE INVENTION

The invention will now be described with reference to examples of preferred formulations known to the inventors.

For the purposes of the examples below, reference will be made to use of green lipped mussel as the mussel extract component in the composition. This should not be seen as limiting as it should be appreciated by those skilled in the art that other types of mussel extract(s) may also be used in accordance with the present invention. Basis for this may be found in the prior art for example the fact that Mytilus edulis has proven anti-inflammatory effects (WO 96/05164).

Further, for the purposes of the examples below, reference will be made to use of carprofen as the NSAID compound in the composition. This should not be seen as limiting as it should be appreciated by those skilled in the art that other types of NSAID compounds may also be used in accordance with the present invention.

### Analysis of Formulations Containing Carprofen and GLME

A group of 50 dogs suffering from osteoarthritis were analysed over a period of 2 weeks during and after treatment with differing amounts of carprofen and mussel extract (kept to either 5 or 8 mg/kg/day). All animals were treated identically other than differing levels of carprofen.

This analysis revealed that doses between 0.5-2.0 mg carprofen per kg per day showed a similar response to the +CTL group (4 mg/kg/day—standard carprofen treatment). However, dosage below 0.5 mg carprofen per kg per day showed a significantly reduced response in both 5 mg and 8 mg mussel extract groups, suggesting that the carprofen was not effective under this carprofen dosage. Furthermore, the 5 mg group showed poorer results than the 8 mg group (and +CTL) when the carprofen was lowered below 2 mg/kg/day. This suggested that the synergistic effect was weakened when the mussel extract was lowered to 5 mg/kg/day. Based on these results, the applicant understands that the threshold is approximately 6 mg/kg/day mussel extract.

The analysis compared the most common side-effects associated with carprofen treatment. The analysis revealed that side-effects were more prevalent in dogs treated with over 2 mg/kg/day than those treated with between 0.5-2 mg/kg/day. Importantly, the prevalence of side-effects seen in the latter group are most similar to the +CTL group (normal carprofen treatment) suggesting that side-effects were substantially abolished when carprofen is reduced to levels below 2 mg/kg/day.

### Preferred Dosages

Based on the trial above, the inventors have provided a summary (Table 2) of the preferred dosage ranges. Dosages may be adjusted largely according to the weight of the animal being treated.

TABLE 1
Carprofen + GLME Tablet Formulation

| | Re-formulation | | | |
|---|---|---|---|---|
| Ingredients | % w/w | PF 7 | PF 9 | PF 10 |
| Carprofen | 5.95 | 50.0 | 20.0 | 100.0 |
| GLME | 33.33 | 280.0 | 112.0 | 559.9 |
| Excipients | | Up to a | Up to a | Up to a |
| | | total of | total of | total of |
| | | 840 mg | 336 mg | 1680 mg |

It should be appreciated that quantities outside of these ranges may also be appropriate depending on other characteristics of the animal including species, breed, age, sex, genetics and physiology of the animal. Dosages may also be taken across different time periods of the day e.g. the dose split in two and half administered in the morning and half in the evening.

The ranges given should also not be seen as limiting as it should be appreciated by those skilled in the art that the composition may also to be used for animals of weights other than 6 kg.

### Dosage

It should be appreciated by those skilled in the art that the on-going dosage level may be largely dependent on the clinical response to treatment by the animal under treatment although it is envisaged that, for the present invention, treatments will continue with a daily dosage taken for a period of one to two months.

Following treatment with the composition of the present invention, it is envisaged that the animal may be further treated with the composition as disclosed in the applicant's related publication, WO 01/05411 for future treatment.

It should be appreciated by those skilled in the art that the above described invention provides an animal treatment for joint disorders that is effective at treating both the symptoms of the disorder as well as the underlying cause of the disorder i.e. cartilage degradation and associated inflammation. It is also envisaged that the combination formulation also acts quickly to treat the disorder, requires a lower dose of COX-2 selective NSAIDs, is more cost effective and has fewer potential side effects.

Aspects of the present invention have been described by way of example only and it should be appreciated that modifications and additions may be made thereto without departing from the scope thereof as defined in the appended claims.

What I/We claim is:

1. A composition for an animal within a particular weight range including at least one non-steroidal anti-inflammatory drug (NSAID) compound and at least one mussel extract
characterised in that
the therapeutic dose of NSAID compound in the composition is substantially less than the usual therapeutic dose.

2. The composition as claimed in claim 1, wherein the therapeutic dose of NSAID compound in the composition is approximately between 0.5-2 milligrams per kilogram of animal per day (mg/kg/day).

3. The composition as claimed in either claim 1 or claim 2 wherein the NSAID is selected from any non-steroidal anti-inflammatory drug.

4. The composition as claimed in claim any one of claims 1 to 3, wherein the NSAID compound is a COX-2 selective NSAID compound.

5. The composition as claimed in claim 4, wherein the COX-2 selective NSAID compound is selected from: carprofen, maproxen, ibuprofen, ketoprofen, piroxicam, diclofenac, etodolac, flunixin, deracoxib, meloxicam, celecoxib, rofecoxib, or combinations thereof.

6. A composition as claimed in claim 4, wherein the COX-2 selective NSAID compound is carprofen.

7. The composition as claimed in any one of claims 1 to 6, wherein the mussel extract is obtained from edible members of the family Perna or Mytilus.

8. The composition as claimed in any one of claims 1 to 7, wherein the mussel extract is obtained from Perna canaliculus.

9. The composition as claimed in any one of claims 1 to 8, wherein the mussel extract is a powdered form of a mussel component not including the shell.

10. The composition as claimed in any one of claims 1 to 9, wherein the extract is a preparation of the lipid portion of the mussel.

11. The composition as claimed in claim 10 wherein the lipid portion of the mussel is prepared by:
a) centrifugation of the lipid portion of the mussel;
b) freeze drying the portion obtained from step (a); and
c) forming a powder from the freeze dried portion.

12. The composition as claimed in any one of claims 1 to 11, wherein the level of mussel extract is approximately between 5-20 times the amount (w/v) of NSAID present in the formulation.

13. The composition as claimed in any one of claims 1 to 12 wherein the amount of mussel extract is approximately 5.6 times the amount (w/v) of NSAID present in the formulation.

14. The composition as claimed in any one of claims 1 to 13, wherein the composition is in a form suitable for oral administration.

15. A method of treatment or prevention of a connective tissue disease or the symptoms thereof in an animal,
characterised by the step of
administration to the animal a composition containing a therapeutically effective amount of at least one NSAID compound and at least one mussel extract,
wherein the therapeutic dose of NSAID compound is substantially less than the usual therapeutic dose.

16. The method as claimed in claim 15 wherein the user:
1) is administered the composition for a period of approximately two weeks;
2) and then is indefinitely administered a stabilizing treatment with less or no NSAID compound than in the composition.

17. The use of a therapeutically effective amount of at least one NSAID compound and at least one mussel extract, wherein the therapeutic dose of NSAID compound is substantially less than the usual therapeutic dose, in the manufacture of a medicament for the treatment or prevention of a connective tissue disease or the symptoms thereof.

18. The method as claimed in any one of claims 15 to 17 with a composition as claimed in any one of claims 1 to 14.

19. The method as claimed in any one of claims 15 to 18 wherein the connective tissue disease to be treated or prevented is selected from osteoarthritis, joint inflammation, cartilage degradation, hip dysplasia, or combinations thereof.

20. The method as claimed in any one of claims 15 to 19 wherein the composition is used to treat both the underlying cause and the symptoms associated with connective tissue disease.

21. The method as claimed in any one of claims 15 to 20 wherein the animal to be treated is a companion animal.

22. The method as claimed in any one of claims 15 to 21 wherein the animal to be treated is from the species canine, feline or equine.

23. The method as claimed in any one of claims 15 to 22 wherein the animal to be treated is a domestic dog.

24. The method as claimed in any one of claims 15 to 23 wherein the composition is used as an effective first line of treatment for connective tissue afflictions including joint inflammation, osteoarthritis and/or cartilage degradation.

25. A composition substantially as herein described and illustrated by the examples excluding prior art disclosed within the specification.

26. A method of treatment or prevention of a connective tissue disease, or the symptoms thereof in an animal, substantially as herein described and illustrated by the examples excluding the prior art disclosed within the specification.

36. The use of at least one NSAID compound and mussel extract substantially as herein described and illustrated by the examples excluding the prior art disclosed within the specification.
